(19) United States
(12) Patent Application Publication
(10) Pub. No.: US 2004/0093624 A1
(43) Pub. Date: May 13, 2004

(54) Method for improving development potential of an embryo and embryos developed therefrom

(76) Inventors: Andrew James French, McKinnon, Victoria (AU); Robert Daniels, South Yarra, Victoria (AU)

Correspondence Address: Fulbright & Jaworski, 666 Fifth Avenue, New York, NY, 10103-3198, US

(21) Appl. No.: 10/333,638

(22) PCT Filed: Jul. 31, 2001
(86) PCT No.: PCT/AU01/00937

(30) Foreign Application Priority Data
Jul. 31, 2000 (AU) PQ9099

(51) Int. Cl. A01K 67/027; C12N 5/08

(52) U.S. Cl. 800/14; 800/15; 800/16; 800/17; 800/18; 435/366

(57) ABSTRACT

The present invention relates to a method of improving development potential of an embryo, embryos developed therefrom and organisms resulting from embryos developed from the method. In a first aspect of the present invention, there is provided a method of culturing an embryo to improve development potential, said method comprising; obtaining an embryo; and culturing the embryo to enhance trophectoderm development of the embryo. The method relates to improving the chances of an embryo implanting to result in a successful pregnancy. The embryos desirably become implantation competent favouring foetal-maternal interaction and development to term of an embryo. The trophectoderm stimulating agent may be any compound which is proven to stimulate normal trophectoderm development. Preferably the agent is fibroblast growth factor-4 (FGF4) protein.

The present invention relates to a method for improving development potential of an embryo, embryos developed therefrom and organisms resulting from embryos developed from the method.

### INTRODUCTION

The implantation and future survival of embryos in utero is dependent upon many factors which contribute to proper development of the embryo to blastocyst stage. After blastocyst development, favourable foetal-maternal interactions contribute to long term survival and maintenance of the embryo/foetus for further development into a successful pregnancy and finally birth of an animal. However, the environment in which the embryo develops is critical for both successful implantation and further development. An understanding of the requirements would enable a higher success rate for pregnancies since natural and artificial implantation techniques are not always 100% possibly due to defective embryos. In nuclear transfer programs, the rate of success in inducing and maintaining pregnancy drops dramatically from that seen in healthy embryos (eg. embryos produced in vivo or in vitro). A large proportion of abnormal pregnancies derived from nuclear transfer embryos are noted and caused by abnormal placental development. This leads to defective foetal-maternal interactions resulting in early in vivo death of the embryo or post natal mortality. This difference in success rate between normal embryos and nuclear transfer embryos, specifically somatic cell cloned nuclear transfer embryos indicates an abnormality in the nuclear transfer embryos or the environment in which the embryos implant and develop.

In normal embryos, it has been found that factors such as fibroblast growth factor 4 (FGF4) are crucial in proper embryo development. Homozygous deletion of the FGF4 gene results in a lethal embryonic phenotype similar to that observed for FGFr2 null mutants. Embryos develop normally to the blastocyst stage but degenerate soon after implantation, apparently due to an inability of the inner cell mass to thrive. In vitro culture of blastocysts demonstrated the absence of any extraembryonic endoderm formation in FGF4 null mutants and, that the mutant phenotype could be rescued by addition of recombinant human FGF4 in the culture medium.

An absence of FGF4 expression and the possibility that other genes from other cell lines involved in embryo development, are also aberrantly expressed in nuclear transfer embryos, may contribute to the low frequency of pregnancy and survival following transfer of cloned blastocysts to recipient animals.

Accordingly, it is an object of the present invention to improve the implantation and development of embryos, particular nuclear transfer embryos.

### SUMMARY OF THE INVENTION

In a first aspect of the present invention, there is provided a method of improving trophectoderm development in a cultured embryo, said method comprising the steps of:

obtaining an embryo

culturing said embryo in the presence of one or more of trophectoderm cells, trophectoderm stimulating agent, cells expressing a trophectoderm stimulating agent or supernatant of a trophectoderm cell culture.

The method relates to improving the chances of an embryo In normal embryos, it has been found that factors such as fibroblast growth factor 4 (FGF4) are crucial in proper embryo development. Homozygous deletion of the FGF4 gene results in a lethal embryonic phenotype similar to that observed for FGFr2 null mutants. Embryos develop normally to the blastocyst stage but degenerate soon after implantation, apparently due to an inability of the inner cell mass to thrive. In vitro culture of blastocysts demonstrated the absence of any extraembryonic endoderm formation in FGF4 null mutants and, that the mutant phenotype could be rescued by addition of recombinant human FGF4 in the culture medium.

An absence of FGF4 expression and the possibility that other genes from other cell lines involved in embryo development, are also aberrantly expressed in nuclear transfer embryos, may contribute to the low frequency of pregnancy and survival following transfer of cloned blastocysts to recipient animals.

Accordingly, it is an object of the present invention to improve the implantation and development of embryos, particular nuclear transfer embryos.

### SUMMARY OF THE INVENTION

In a first aspect of the present invention, there is provided a method of culturing an embryo to improve development potential, said method comprising:

obtaining an embryo; and

culturing the embryo to enhance trophectoderm development of the embryo.

The method relates to improving the chances of an embryo implanting to result in a successful pregnancy. The embryos desirably become implantation competent favouring foetal-maternal interaction and development to term of an embryo.

In yet another aspect of the present invention, there is provided a method of developing an animal, said method comprising:

obtaining an embryo with improved development potential and prepared by the methods described above;

obtaining a receptive animal capable of incubating an embryo to term;

implanting the embryo into the receptive animal; and

allowing the receptive animal to incubate the embryo to term.

In another aspect of the present invention, there is provided an animal obtained by the methods described.

### IN THE FIGURES

FIG. 1 shows trophectoderm cell line resides with underlying monolayer.

FIG. 2 shows RT-PCR results of fibroblast (F), term placenta (Pl) and TE cells.

### DESCRIPTION OF THE INVENTION

In a first aspect of the present invention, there is provided a method of culturing an embryo to improve development potential, said method comprising:

obtaining an embryo; and

culturing the embryo to enhance trophectoderm development of the embryo.

The method relates to improving the chances of an embryo implanting to result in a successful pregnancy. The embryos desirably become implantation competent favouring foetal-maternal interaction and development to term of an embryo.

Applicants have found that FGF4 expression is aberrant in a high proportion of embryos derived from somatic cell nuclear transfer techniques. This coincides with the absence of viable trophectoderm cell lineages from blastocyst stage mouse embryos lacking the FGF4 gene. These deficiencies correlated with an observed higher proportion of abnormal pregnancies from nuclear transfer

The trophectoderm cell lineage is considered to be important for successful implantation and further survival of the mammalian embryo in utero. However as recently found by the applicants, some embryos which result in abnormal pregnancy and/or abnormal placental development have a tendency to have deficient FGF4 expression and possibly aberrantly expressed genes involved in trophectoderm development.

Targeting the trophectoderm or enhancing its development may be achieved by exposure of the embryo to normal trophectoderm either directly, or indirectly, or through exposure of the embryo to a trophectoderm stimulating agent.

Alternatively, the embryo may be exposed to supernatant of a trophectoderm cell culture.

In a preferred aspect, the method includes the steps of:

obtaining a source of trophectoderm cells; and

culturing the embryo in the presence of the trophectoderm cells.

The trophectoderm cells may be derived from any source but preferably the source is compatible to the embryos that are being cultured.

The trophectoderm cells may be a cell line derived from trophectoderm cells of any species. Preferably such trophectoderm cells will be derived from the same species as the embryo. The term “trophectoderm cells” as used herein is intended to include all types of trophectoderm cells including “mature” trophectoderm cells, trophectoderm stem cells, trophectoderm vesicles or trophectoderm like cells identifiable by the expression of growth factors selected from the group including but not limited to TP, FGFr-2, LIF, EGF, HB-EGF or EGFR.

The trophectoderm cells may be derived from the embryo itself to create a trophectoderm cell monolayer. Preferably, the trophectoderm cells are a normal trophectoderm cell derived from a healthy source.

Without being limited by theory, it is possible that the aberrant development of the trophectoderm lineages in embryos, particularly nuclear transfer embryos may be corrected if the nuclear transfer embryos were cultured in the presence of normal trophectoderm cells preferably prior to transfer to a recipient animal.

The trophectoderm cell lines may provide factors in the media that would support the normal foetal placental development. It is postulated that the trophectoderm cells may be male or female and derived from in vitro or in vivo produced embryos. They may be bovine, for bovine nuclear transfer embryos, but the trophectoderm cells from any species could be matched with the nuclear transfer embryos from another species. The trophectoderm lineages may be isolated as previously described (Tanaka et al., 1998; Flechon et al., 1995) or using alternative methods known to the addressee.

The trophectoderm cells may be present as a cell culture, preferably a monolayer or as a cell suspension or they may be trophoblast vesicles from in vitro or in vivo produced embryos. In this preferred aspect, the presence of the trophectoderm cells enhances the development of the trophectoderm cells or the embryo. The trophectoderm cells may be placed in close proximity to the embryo or be aggregated with the embryo either by placement of trophectoderm cells on the embryo, such as in the absence of the zona pellucida or they may be placed under the zona pellucida when the zona pellucida is present.

The embryo may be cultured to the blastocyst stage or to any stage where trophectoderm development of the embryo is enhanced for favourable implantation and placenta development. The embryo may be cultured to any stage of development. Preferably, the embryo is transferred preferably onto a monolayer of trophectoderm cells at day 5 of preimplantation development or the morula stage equivalent for any species, and cultured further to the blastocyst stage. However, the embryo may be transferred preferably onto the trophectoderm monolayer at any stage of preimplantation development.

In a further preferred aspect, there is provided a method of culturing an embryo to improve development potential, said method comprising:

obtaining an embryo at the blastocyst stage;

obtaining a source of trophectoderm cells; and

introducing the trophectoderm cells into the blastocyst to provide an embryo suitable for culturing or implantation.

The embryo may be as described above and cultured to a blastocyst stage by any methods known to the skilled addressee. Preferably, the blastocyst stage is a stage where the blastocyst cavity has developed.

The embryo may be any mammalian embryo but preferably it is a nuclear transfer embryo derived by any nuclear transfer method available to the addressee, using any cell type as the source of the donor nucleus. The embryo culture system used may be any culture system capable of supporting the successful development of nuclear transfer embryos to the blastocyst stage.

Similarly, the trophectoderm cells may be as described above and cultured by any methods known to the skilled addressee. Specifically, such trophectoderm cells may be derived from a trophectoderm cell line or isolated as trophoblast vesicles from in vitro or in vivo produced embryos. Such trophectoderm cells may be derived from any species. However, it is preferred that the trophectoderm cells will be derived from the same species as the embryo. For instance, bovine trophectoderm cells will be used for bovine embryos, but it is also within the scope of the invention to use trophectoderm cells from any species to inject into embryos of other compatible species.

The trophectoderm cells may be injected into the cavity of blastocyst stage embryos. The injected trophectoderm cells may contribute to the extraembryonic cell lineages and may help support the development of embryos, particularly nuclear transfer embryos, specifically the extraembryonic cell lineages.

The trophectoderm cells may be injected into the blastocyst cavity by any of the methods available which do not harm the embryo. Micromanipulation is preferred.

The number of trophectoderm cells may be varied. However, it is preferred to inject from 1 to 100 trophectoderm cells into the blastocyst cavity.

Alternatively, the trophectoderm cells may be introduced into the blastocyst by aggregating trophectoderm cells with the embryo by either placing trophectoderm cells on the embryo (in the absence of zona pellucida) or inserting trophectoderm under the zona pellucida when the zona pellucida is present. This allows the trophectoderm cells to integrate with the embryo.

In a further preferred aspect, the method further includes the step of:

culturing the embryo preferably to the hatching blastocyst stage or any stage of blastocyst development.

The further culturing period will depend on the preferred stage of development of the blastocyst and also of the species of embryo cultured. However, any period of 24 to 48 hours is preferable. After this period, the injected embryo may be transferred to a recipient animal.

In yet another preferred aspect of the invention, the method further includes the step of:

transferring the embryo after introduction of the trophectoderm cells to a recipient animal.

Any methods of transfer are available to the skilled addressee. However, general IVF techniques are suitable.

In a further preferred aspect of the present invention, there is provided a method of culturing an embryo to improve development potential, said method comprising:

obtaining an embryo; and

culturing the embryo in the presence of a trophectoderm stimulating agent.

The trophectoderm stimulating agent may be any compound which is proven to stimulate normal trophectoderm development. Preferably the agent is fibroblast growth factor-4 protein (FGF4) either in its natural or recombinant form, wherein the recombinant form is added extrinsically or produced in-situ. The FGF-4 may also be derived from cell cultures. Preferably, FGF-4 is provided in the supernatant of an embryonic carcinoma cell (ECC) culture.

Fibroblast growth factor 4 (FGF4) has previously been shown to be essential for the isolation of trophectoderm cell lines from mice and pigs. In addition, in the mouse, the aberrant developmental phenotype of FGF4 homozygous mutant embryos in vitro has been reversed by the addition of FGF4 to the culture media (Feldman 1995). However, despite the high number of reports of placental abnormalities and low implantation rates for embryos produced by nuclear transfer techniques and, the applicants recent finding that a large percentage of nuclear transfer embryos aberrantly express FGF4 at the blastocyst stage, there are no reports in the literature regarding the use of FGF4, or any other growth factors, in the culture media of nuclear transfer-derived embryos in an attempt to correct the apparently abnormal development of the extraembryonic cell lineages.

The embryo is as described above. Preferably, the embryo has been cultured to the morula stage or the blastocyst stage prior to addition of the trophectoderm stimulating agent. Preferably, the embryo is at the morula stage.

The trophectoderm stimulating agent or combination of agents may be added to an embryo culture at a suitable time of development of the embryo such as the morula or blastocyst stage, or the media may be changed to one already containing the trophectoderm stimulating agent. The time for changing the media or introducing the trophectoderm stimulating agent will vary. However, it is preferred to introduce the trophectoderm stimulating agent or combination of agents at approximately day 5 or at the morula stage equivalent depending on the species of animal.

Where recombinant trophectoderm stimulating agent is used, for instance recombinant FGF4 preferably FGF4 in the presence of heparin, the origin is preferably compatible with the species of embryo used. For instance for bovine embryos, bovine recombinant trophectoderm stimulating agent or preferably bovine FGF4 is used. However, recombinant FGF4 protein derived from any species could be used with embryos from any other species dependent on cross species reactivity.

The amount of trophectoderm stimulating agent used will depend on the species. However a concentration of 15 to 25 ng/ml preferably 20 ng/ml is used for addition to morula stage embryos.

In another aspect of the present invention, there is provided an embryo produced by the methods described. Preferably, the embryo is a blastocyst.

The embryo, blastocyst or any stage of embryo development may be nuclear transfer derived. These may be further cultured to a stage of hatching demonstrating a level of implantation competency. Accordingly, in a preferred aspect, there is provided an embryo, blastocyst or any stage of embryo development ready for implantation.

It is also conceivable to use a genetically modified embryo, wherein the embryo is modified to express a trophectoderm stimulating agent such as FGF4. The embryo may be modified at any stage, preferably prior to fertilization at the oocyte and gamete stage. The oocyte or gamete may have introduced constructs which can express a trophectoderm stimulating agent, preferably FGF4. Enhanced expression may ensure improved development potential. Methods to enhance expression of trophectoderm stimulating factor activity may be achieved by any recombinant means so as to achieve trophectoderm development of the embryo. Suitable recombinant constructs incorporated into genetically modified embryos may allow the activation of expression of trophectoderm stimulating agents at appropriate times to improve development potential.

In yet another aspect of the present invention, there is provided a method of developing an animal, said method comprising:

obtaining an embryo with improved development potential and prepared by the methods described above;

obtaining a receptive animal capable of incubating an embryo to term;

implanting the embryo into the receptive animal; and

allowing the receptive animal to incubate the embryo to term.

The embryo may be a blastocyst or be at any stage of embryo development providing it has been prepared by the methods described herein.

The receptive animal is an animal capable of carrying a foetus to term and may be a female animal in a breeding cycle or artificially induced to accept an embryo and to carry the foetus to term. By “artificially induced” it is meant that pharmaceutical grade synthetic hormones such as follicle stimulating hormone (FSH) in conjunction with luteinizing hormone (LH), using prescribed stimulation protocols for a given species, be injected in to the animal to prepare the womb for receiving the blastocyst

In another aspect of the present invention, there is provided an animal obtained by the methods described.

The procedures described herein are designed to produce embryos, particularly nuclear transfer embryos with an improved capability of implantation in recipient animals and ultimately an improved efficiency of producing viable cloned animals. The procedures described have the advantage of producing embryos, particularly nuclear transfer embryos with an improved trophectoderm cell lineage with an increased chance of producing a viable extraembryonic cell lineage capable of normal implantation events, normal foetal/maternal interactions and capable of producing a placenta able to provide sufficient support to the developing foetus.

Throughout the description and claims of this specification, the word “comprise” and variations of the word, such as “comprising” and “comprises”, is not intended to exclude other additives, components, integers or steps.

Examples of the procedures used in the present invention will now be more fully described. It should be understood, however, that the following description is illustrative only and should not be taken in any way as a restriction on the generality of the invention described above.

### EXAMPLES

### Example 1

Analysis of FGF4 Xpression in Bovine Nuclear Transfer Embryos

(a) Collection of Bovine Oocytes

Bovine ovaries were obtained from a local slaughterhouse, transported at 25-30° C. to the laboratory and washed in warmed phosphate buffered saline (PBS, Baxter, Australia). Ovarian antral follicles (2-8mm) were aspirated using an 18-gauge needle and collected into Hepes buffered Tissue Culture Medium 199 (TCM199, Gibco BRL/Life Technologies) with heparin (5000 iu/ml, Sigma), 2% Foetal Calf Serum (FCS, Gibco/Life Technologies), and amphotericin B (250μg/ml, Sigma). Cumulus oocyte complexes (COC's) showing an even cytoplasm and surrounded by at least three layers of compact cumulus cells were collected from the follicular fluid. COC's were incubated and matured in groups of 25 in a TCM199 medium supplemented with gentamycin sulfate (10 mg/ml), L-glutamine (29 mg/ml, Sigma), human Chorionic Gonadotrophin (1500 lU/ml, Lyppards, Australia) and 15% FCS at 39° C. in 5%CO₂ in air, for 20-24 hours.

(b) Preparation of Oocytes for Nuclear Transfer

In order to remove the surrounding cumulus, matured oocytes at 19-21 hours post maturation (hpm) were vortexed in 80 μl maturation media and 20 μl hyaluronidase (0.1%, Sigma) for 3 minutes in Eppendorf tubes (Quantum Scientific). The oocytes were washed through handling media (Hepes buffered TCM199 with 5% FCS (199HF)) and those at the metaphase II stage (i.e. with the first polar body extruded) were selected for nuclear transfer (NT).

(c) Fibroblast Cell Collection and Culture

Fibroblast cells were prepared from skin and muscle sections from approximately 50-60 day old bovine foetuses. Tissue sections were diced in PBS using sterile scalpels and tweezers prior to digestion in 0.25% trypsin at 37° C. for 20-30 minutes. DMEM culture media containing 10% FCS was then added to the sample to inactivate the trypsin and, the sample centrifuged for 5 minutes to pellet the cells. Following the removal of the supernatant, the cells were resuspended in DMEM with 10% FCS and cultured for up to three passages. Prior to nuclear transfer, fibroblast cells at 70% confluency were cultured for a further 5-7 days in serum depleted media (DMEM plus 0.5% FCS).

(d) Granulosa Cell Collection and Culture

Mural granulosa cells were collected from an elite superovulated calf using an ultrasound-guided transvaginal probe. Granulosa cells were present in the collection media (DMEM containing 20 μg/ml Amphotericin B, 1 mg/ml Kanomycin Sulphate, 40 μg/ml Chloramphenicol, 100 μg/ml Chlorotetracycline, 60 μg/ml Penicillin and 100 μg/ml Streptomycin, Sigma) as morphologically distinct cell sheets. Granulosa cell sheets were placed on a percoll gradient (Sigma) using a bi-layer of 50% and 25% percoll, and centrifuged at 600G for 20 minutes. Cells located at the interface were collected and washed twice in DMEM with 10% FCS. Granulosa cells were cultured in DMEM with 10% FCS for up to three passages. Prior to nuclear transfer, granulosa cells at 70% confluency were cultured for a further 5-7 days in serum depleted media (DMEM plus 0.5% FCS).

(d) Nuclear Transfer by Microinjection

After mechanical disruption of the donor cell membranes in 199HF using the injection pipette, mural granulosa cells were injected directly into the cytoplasts. The reconstructed embryos were transferred back into TCM199+10% FCS until activation.

(e) Nuclear Transfer by Cell Fusion (SUZI)

Bovine oocytes were enucleated at 18-22 hpm in handling media containing cytochalasin B (7.5 μg/ml, Sigma) by gentle aspiration of the polar body and metaphase plate in a small amount of cytoplasm using a glass pipette (inner diameter: 10-15 μm). A donor cell is then injected into the oocytes perivitelline space, directly following enucleation. The oocyte-cell complexes are cultured in maturation medium for approximately half an hour to one hour prior to cell fusion. Oocyte-cell complexes are tranferred to mannitol fusion media at room temperature, aligned at 600 KHz pre 6.0 V AC and fused with two pulses of 80.0-90.0 V DC for 15-30 μs, one second apart, using wire electrodes 0.5 mm apart. The oocyte-cell complexes are then placed into the maturation medium to allow cytoplasmic fusion to occur (5-20 minutes).

Artificial activation was induced either 0.5 or 4 hours after fusion or injection by exposing the oocytes to 5 μM calcium ionophore for 4 minutes, prior to culture in 2 mM 6-DMAP for five hours.

Embryos were cultured in modified Synthetic Oviductal Fluid (SOF) culture media (Gardner et al, 1994) supplemented with amino acids (Sigma), 5% FCS, myo-inositol (0.05 g/10 ml, Sigma) and sodium tri citrate (1 mg/1 ml, Selby Scientific). Embryos were submerged in a Submarine-lncubation-System (SIS, Vajta et al, 1997). The 4-well plates were gassed in foil bags (Wests Packaging Services) with 5% O₂, 5% CO₂ and 90% N₂ and immersed in 39° C. water for up to seven days.

(f) Reverse Transcriptase Polymerase Chain Reaction (RT-PCR) Embryo Analysis

The protocols used for sample preparation, reverse transcription (RT) and polymerase chain reaction (PCR) amplification have been described previously (Daniels et al, 1997). Briefly, single oocytes or embryos were added to 5 μl lysis buffer (0.8% Igepal, 5 mM DTT, 1 U/μl RNA sin), snap frozen in liquid nitrogen and stored at −80° C. prior to use. When required, samples were heated to 80° C. for 5 minutes, transferred straight to ice and the RT premix added. Reverse transcription was carried out in a final volume of 10 μl comprising of the cell lysate, 1×RT Buffer, 100 U SuperScript H⁻ reverse transcriptase (GIBCO, BRL), 1.5 μg random primers (GIBCO, BRL), 5 mM DTT and 1 U/μl RNA sin. Reactions were held at 37° C. for one hour. For granulosa cell samples, cells were scraped from the culture flask and pelleted in an Eppendorf tube in STE buffer (0.1 M NaCl, 20 mM Tris pH 7.4, 10 mM EDTA pH 8.0). The supernatant was then removed, the cells resuspended in 40 μl lysis buffer, snap frozen in liquid nitrogen and stored at −80° C. On use, cell lysates were thawed and centrifuged at 12000 g for 10 minutes to pellet cell debris. The supernatant was then transferred to a fresh Eppendorf tube and mRNA was extracted using a Dynal Beads mRNA purification kit (Dynal Pty. Ltd., Australia), as directed. Reverse transcription was carried out in a 20 μl reaction mix with reagent concentrations as described for embryo analysis. Negative controls, omitting reverse transcriptase or added sample were always included.

PCR amplification was carried out on 2.5 μl of the RT product from embryos or 1 μl (approximately 20 ng RNA or 2000cells equivalent) from granulosa cell cDNA products. PCR cycles were as follows: 94° C.×5′ followed by 50 cycles for embryos or 30 for cell samples of 94° C.×1′; 52° C.×1′; 72° C.×2′. Ten microlitres of the PCR products were visualised under ultra violet light on 2% agarose gels containing 1 μg/ml ethidium Bromide. The PCR primer sequences for FGF4 were (5′ to 3′) TTCTTCGTGGCCATGAGCAG and AGGAAGTGGGTGACCTTCAT.

Results

In an initial experiment on nuclear transfer embryos derived from the microinjection of granulosa cell nuclei followed by activation of the resulting embryos 0.5 hrs after nuclear transfer, FGF4 transcripts were detected in only two of the nine embryos analysed at the morula and blastocyst stages. This is a significantly lower number (p<0.01) when compared to the detection of FGF4 transcripts in all ten IVF embryos analysed.

In a second experiment, a number of nuclear transfer embryos reconstructed with fibroblast nuclei were analysed for the presence of FGF4 transcripts. Embryos were produced by either microinjection and artificial activation either 0.5 (Group A) or 4 (Group B) hours after injection or cell fusion (SUZI) and activation 4 hours after fusion (Group C). In IVF embryos analysed at the blastocyst stage, FGF4 transcripts were detected in 37/43 embryos analysed (86%). However, FGF4 transcripts were detected in significantly fewer embryos at the blastocyst stage in group A (8/21, 38%, p<0.0005) and, in fewer embryos but with no significant difference in groups B (12/20, 60%) and C (13/21, 62%).

The results indicate that FGF4 is aberrantly expressed in a large proportion of nuclear transfer embryos produced with different donor cell nuclei and with different nuclear transfer techniques. Aberrant expression of FGF4 could indicate the abnormal development of the trophectoderm lineage.

### Example 2

Trophectoderm Enhancement of Nuclear Transfer Embryos

In order to assess the potential value of supporting the development of the trophectoderm lineage, embryos produced using SUZI nuclear transfer procedures, artificial activation 4 hours after fusion and fibroblast cells as the source of the donor nuclei, as described above, were separated into four groups. A control group of embryos were cultured to the day 7 blastocyst stage as described above and, three experimental groups were treated as described below.

1) Culture of Embryos on a Monolayer of Trophectoderm Cells.

At day 5 of culture, embryos at the morula stage of development were transferred in SOF media onto a monolayer of trophectoderm cells. Trophectoderm lineages were isolated from in vitro fertilised bovine embryos at the blastocyst stage as previously described (Tanaka et al., 1998; Flechon et al., 1995). The embryos were cultured for a further 48 hours prior to transfer to recipient cows.

2) Injection of Trophectoderm Cells into Blastocyst Cavity.

Day 6 embryos at the early blastocyst stage had approximately 10 trophectoderm cells injected in to the blastocyst cavity. The trophectoderm cells were isolated from a trophectoderm cell lineage as described above. The embryos were cultured for a further 24 hours before being transferred into recipient cows.

3) Addition of Recombinant FGF4 to Embryo Culture Media.

At day 5 of culture, human recombinant FGF4 protein was added to the culture medium of embryos at the morula stage of development to a final concentration of 20 ng/ml. The embryos were cultured for a further 48 hours prior to transfer to recipient cattle.

For each of the three experimental groups of embryos, ten recipient cows received two blastocyst stage embryos each. The cows were pregnancy tested at day 30 and day 60 using ultrasound techniques.

### Example 3

Blastocyst Development and Differential Staining of Bovine Embryos Treated with rhFGF4

a) Isolation and Culturing of Trophectoderm (TE) Cells.

Blastocysts (9 day IVP-produced) were primarily seeded on bovine fibroblast feeder cells and subsequent cultures were grown on 1% gelatin layers, cultured in Dulbecco's Modified Eagles medium (Trace Biosciences) supplemented with L-glutamine (Gibco BRL, Invitrogen), sodium bicarbonate (BDH), 100 lU/ml penicillin (Gibco BRL, Invitrogen), 100 μg/ml streptomycin, 1% non essential amino acids (Sigma Chemical Co.), 15% FCS (Gibco, BRL Invitrogen), 1 mg/ml heparin (Sigma Chemical Co.) and 15% conditioned medium, which was collected from the supernatant of embryonic carcinoma cells (ECC), known to secrete fibroblast growth factor-4 (FGF-4). ECC supernatant was found to be a useful source of FGF4. The use of 1% gelatin is a simple and effective feeder layer, forming an appropriate membrane for attachment and proliferation of the cells, which did not require the use of cell feeder layers.

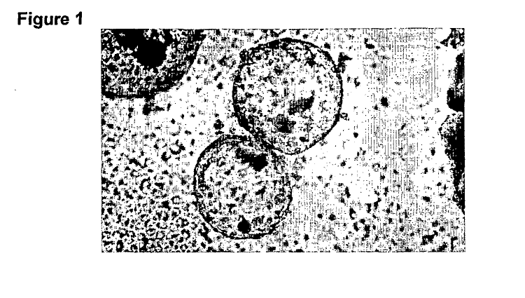

Two weeks after initial attachment, cells were passaged by standard trypsinisation or mechanical lifting. Following one month in culture, TE cells were passaged weekly by mechanically lifting of the monolayers and vesicles. Morphological analysis of cultures suggested that following attachment of blastocysts to bovine feeder layers, ICM cells degenerated and trophectoderm cells grew as monolayers of epithelium with dome-like formations from the centre of most colonies (see FIG. 1). Trophectoderm vesicles were abundant during culture, varying in size and appearing morphologically like enlarged embryos. Differentiation of trophectoderm cells into giant cells was noticeable at the periphery of some colonies, however, initial culture experiments showed that differentiation appeared to be less prominent with the addition of conditioned medium containing FGF4. Pure cultures of TE cells were isolated and have been grown continuously for 6 months up to passage 13.

b) Cryopreservation

Trophectoderm cell lines were successfully frozen and thawed when vesicles were vitrified using standard Open-Pulled-Straw procedures. Viability of trophectoderm cell lines using standard cell freezing was extremely low.

c) Characterisation

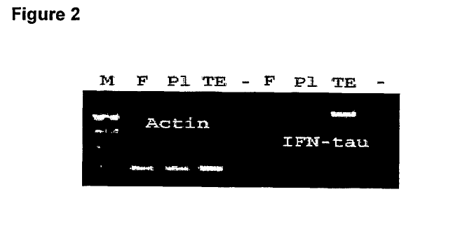

TE cells were identified by expression of interferon-tau (IFN-τ) gene transcripts. IFN-τ was expressed in trophectoderm cells, as it is responsible for maternal recognition of pregnancy in the bovine, with expression highest at day 12-15 of development. Results were compared against actin expression as shown in FIG. 2.

d) Addition of FGF4 to IVP Embryos

The following experimental treatments were added on day 5 after fertilisation of embryos to coincide with expression of FGF4.

I SOF+CS

II SOF+BSA

SOF+CS plus rhFGF4 (20 ng/ml, rhFGF4, and 1 mg/ml heparin, Sigma Chemical Co.)

IV SOF+BSA plus rhFGF4

V SOF+BSA plus 5% CMed (conditioned medium of ECC cultures) and 5% FCS

VI SOF+BSA plus rhFGF4 and 5% c/tFCS (charcoal treated foetal calf serum)

VII SOF+BSA plus 5% c/tFCS

VIII SOF+BSA plus 5% CMed and 5% c/tFCS

Embryos (IVP—In vitro produced bovine embryos) were cultured for 7 days in SOFM before being differentially stained for TE:ICM Ratios.

Table 1 shows that resuls of blastocyst development and differential straining of bovine embryos treated with rhFGF4.

TABLE 1
Blastocyst development and differential staining of bovine embryos treated with rhFGF4 and CMed.

| | Blastocysts | | Differential Staining | | Total Cell. | Ratio |
|---|---|---|---|---|---|---|
| Treatment Groups | (%) | No. | ICM cell no. | TE cell no. | No. | ICM:TE |
| I). IVP CS | 254/927 (27) | 23 | 34.3 ± 2.20^{b} | 117.0 ± 7.88^{b} | 151.3 ± 8.26^{a} | 1:3.4 |
| II). IVP BSA | 201/814 (25) | 23 | 33.70 ± 2.87^{b} | 110.8 ± 5.12^{b} | 144.5 ± 6.54^{b} | 1:3.2 |
| III). IVP CS:rhFGF4 | 215/887 (24)^{a} | 19 | 28.16 ± 2.23^{b} | 97.21 ± 5.19 | 125.4 ± 6.48^{a} | 1:3.5 |
| IV). IVP BSA:rhFGF4 | 251/865 (29)^{b} | 31 | 32.96 ± 2.09^{b} | 98.48 ± 4.63 | 127.5 ± 5.04^{a} | 1:3.0 |
| V). IVP BSA:CMed+FCS | 149/573 (26) | 20 | 35.85 ± 2.14 | 95.85 ± 5.28^{a} | 132 ± 5.38 | 1:2.7 |
| VI). IVP BSA:rhFGF4+c/tFCS | 127/399 (32)^{b} | 35 | 37.97 ± 1.49 | 94.77 ± 2.54^{a} | 132.8 ± 3.32 | 1:2.5 |
| VII). IVP BSA:c/tFCS | 225/713 (32)^{b} | 21 | 36.81 ± 2.02 | 108.3 ± 5.45 | 145.1 ± 6.87^{b} | 1:2.9 |
| VIII). IVP BSA:CMed+c/tFCS | 288/1002 (29)^{b} | 22 | 44.50 ± 3.14^{a} | 102.0 ± 3.47 | 146.5 ± 5.15^{b} | 1:2.3 |

Comparison between a vs b within columns are significantly different (p < 0.05)

Results show the following:

The ratio of ICM:TE in all treatment groups is approximately 1:3.0. Addition of (VII) CMed and c/tFCS on day 5 resulted in significantly higher numbers of ICM cell (p<0.05) when compared to control and rhFGF4 treatment groups (I, II, III and IV).

Addition of FGF4 (either with rhFGF4 or CMed) appeared not to increase TE proliferation, and in groups (V and VI) was significantly lower (P<0.05) when compared to controls (I and II). However, addition of FGF4 or CMed may provide conditions that establish a tighter control over the ratio of ICM:TE (Reports show that the developmental competence of a transferred blastocyst is related to the establishment of a ICM:TE ratio of 1:3).

Initial testing of in vivo development following embryo transfer indicated embryos from groups I, II, III, IV and VII transferred to recipients (2 embryos per recipient) and assessed for pregnancy by ultrasound between day 30 and 60 resulted in 24/53(45%), 12/20(60%), 4/10(40%), 6/9(67%) and 5/7(71%) pregnancy rate, respectively. It appears that addition of rhFGF4 or CMed does not have a detrimental effect on embryo implantation.

Finally, it is to be understood that various other modifications and/or alterations may be made without departing from the spirit of the present invention as outlined herein.

### REFERENCES

Daniels et al., 1997. Human Reproduction 12: 2251-2256.

Flechon et al., 1995. Placenta 16:643-658

Gardner et al., 1994. Biology of Reproduction 50:390-400.

Tanaka et al., 1998. Science 282:2072-2075

Vajta et al., 1997. Theriogenology 48: 1379-1385.

1. A method of culturing an embryo to improve development potential, said method comprising:
obtaining an embryo; and
culturing the embryo to enhance trophectoderm development of the embryo.

2. A method according to claim 1 wherein the embryo is selected from the group including naturally conceived embryos, artificially fertilised embryos, nuclear transfer embryos, cloned nuclear transfer embryos or genetically modified embryos.

3. A method according to claim 1 or 2 wherein the embryo is derived from a source selected from the group including bovine, ovine, porcine, caprine, murine or human.

4. A method according to any one of claims 1 to 3 wherein the embryo is a nuclear transfer embryo.

5. A method according to any one of claims 1 to 4 wherein the trophectoderm development is enhanced by exposure of the embryo directly or indirectly to trophectoderm cells, exposure of the embryo to a trophectoderm stimulating agent, or exposure to a supernatant of a trophectoderm cell culture.

6. A method according to any one of claims 1 to 5 further including the steps of:
obtaining a source of trophectoderm cells; and
culturing the embryo in the presence of the trophectoderm cells.

7. A method according to claim 6 wherein the trophectoderm cells are selected from the group including mature trophectoderm cells, trophectoderm stem cells, trophectoderm vesicles or trophectoderm like cells identifiable by the expression of growth factors selected from the group including TP, FGFr-2, LIF, EGF, HB-EGF or EGFR.

8. A method according to claim 6 or 7 wherein the trophectoderm cells are cultured as a monolayer or as a cell suspension with the embryo.

9. A method according to claim 6 or 7 wherein the trophectoderm cells are aggregated with the embryo.

10. A method according to claim 9 wherein the trophectoderm cells are placed on the embryo.

11. A method according to claim 9 wherein the trophectoderm cells are cultured under the zona pellucida of the embryo.

12. A method according to any one of claims 1 to 11 wherein the embryo is developed to a morula stage.

13. A method according to any one of claims 1 to 11 wherein the embryo is developed to a blastocyst stage.

14. A method according to claim 13 wherein the trophectoderm cells are introduced into the blastocyst by injecting into the blastocyst cavity.

15. A method according to claim 14 wherein at least one trophectoderm cell is injected into the blastocyst cavity.

16. A method according to any one of claims 1 to 15 wherein the embryo is cultured to a hatching blastocyst stage.

17. A method according to any one of claims 1 to 16 wherein the embryo is cultured in the presence of a trophectoderm stimulating agent.

18. A method according to claim 17 wherein the trophectoderm stimulating agent is fibroblast growth factor-4 protein (FGF-4).

19. A method according to claim 18 wherein the FGF-4 is natural or recombinantly produced.

20. A method according to any one of claims 17 to 19 wherein the trophectoderm stimulating agent is cultured with the embryo at a stage equivalent to the morula stage of development.

21. A method according to any one of claims 17 to 20 wherein the trophectoderm stimulating agent is present at a concentration of 15 to 25 ng/ml.

22. A method according to any one one of claims 17 to 21 wherein the embryo is a genetically modified embryo which is modified to express FGF-4.

23. A method according to any one of claims 1 to 22 wherein the embryo is cultured for a period of at least 24 hours prior to transferring to a receptive animal.

24. An embryo with improved development potential prepared by a method according to any one of claims 1 to 23.

25. A method of developing an animal, said method comprising:
obtaining an embryo with improved development potential according to claim 24;
obtaining a receptive animal capable of incubating an embryo to term;
implanting the embryo into the receptive animal; and
allowing the receptive animal to incubate the embryo to term.

26. A method according to claim 25 wherein the receptive animal is a female animal in a breeding cycle or is artificially induced.

27. An animal prepared by the method according to claim 25 or 26.

28. A method according to claim 1 substantially as hereinbefore described with reference to the examples.

26. An animal prepared by a method according to claim 24, or 25.

27. A method of determining embryo viability comprising the steps of:
(i) measuring expression level and/or expression timing of FGF-4; and
(ii) comparing expression level and/or expression timing to expression level and/or expression timing of an in vitro fertilised embryo, wherein embryos having similar expression levels and/or expression timing have greater viability.

28. A method of screening for compounds capable of improving trophectoderm development comprising the steps of:
(i) obtaining an embryo
(ii) culturing said embryo, in the presence of one or more compounds suspected of improving trophectoderm development; and
(iii) comparing the ratio of inner cell mass cells to trophectoderm of the embryo from step (ii) as compared to untreated embryos.

29. A compound obtained by a method according to claim 29.
